(19) United States
(12) Patent Application Publication
(10) Pub. No.: US 2017/0027643 A1
(43) Pub. Date: Feb. 2, 2017

(54) TRANSLUCENT CANNULA WITH SELF CONTAINED COOLING FUNCTION AND ENHANCED VISIBILTY FOR ABLATION CATHETER

(71) Applicant: 1389251 Ontario Inc., Toronto (CA)

(72) Inventor: John Trachtenberg, TORONTO (CA)

(73) Assignee: 1389251 Ontario Inc., Toronto (CA)

(21) Appl. No.: 14/811,833

(22) Filed: Jul. 29, 2015

(51) Int. Cl. A61B 18/22; A61B 34/00; A61B 34/30; A61M 5/00; A61B 34/20

(52) U.S. Cl. CPC A61B 18/22; A61M 5/007; A61B 34/20; A61B 34/30; A61B 34/70; A61B 2018/00029

(57) ABSTRACT

A component for use in magnetic resonance image-guided laser ablation has:
d) a one-piece cannula having at least one laser-transmitting fiber fixed thereto;
e) the one-piece cannula comprising a composition having a proximal insertion end and a thermal energy-emitting tip; and
f) fluid conducting channels fixed to the energy-emitting tip.

The fluid conducting channels have fluid-carrying dimensions sufficient to transport sufficient liquid at 15° C. through the channels to cool both tissue adjacent the channels and the tip during emission from a tissue ablating laser within the thermal energy emitting tip. The composition of the one-piece cannula is at least translucent/transparent to at least 50% of infrared radiation between 900-1200 nm emitted from within the cannula at the thermal energy-emitting tip. The composition of the one-piece cannula should have a melting temperature of at least 150° C.

### BACKGROUND OF THE INVENTION

1. Field of the Invention

The present invention relates to the field of medical technology, and especially laser ablation technology, including both instrumentality and methods.

2. Background of the Art

The delivery of radio frequency (RF) energy to target regions within tissue is known for a variety of purposes of particular interest to the present inventions. In one particular application, RF energy may be delivered to diseased regions (e.g., tumors) in tissue for the purpose of tissue necrosis. RF ablation of tumors is currently performed within one of two core technologies.

The first technology uses a single needle electrode, which when attached to a RF generator, emits RF energy from the exposed, uninsulated portion of the electrode. This energy translates into ion agitation, which is converted into heat and induces cellular death via coagulation necrosis. The second technology utilizes multiple needle electrodes, which have been designed for the treatment and necrosis of tumors in the liver and other solid tissues. PCT Publication WO 96/29946 and U.S. Pat. No. 6,379,353 disclose such probes.

Whichever technique is used for treatment, the target site, e.g., the tumor, is often dangerously close to vital organs or tissue (e.g., colon, prostate, gall bladder, or diaphragm). In many cases, to prevent or reduce the risk of thermally injuring the vital organs or tissue, the physician will opt to discontinue the procedure, or prematurely stop the procedure, resulting in a high likelihood of re-occurrence.

Thus, there is a need for an improved system and method for protecting vital organs or tissue from thermal damage that may otherwise result during ablation of adjacent tissue.

Published U.S. Patent Application Document No. 20150005719 (Fischell) describes an intravascular catheter for peri-vascular and/or peri-urethral tissue ablation includes multiple needles advanced through supported guide tubes which expand around a central axis to engage the interior surface of the wall of the renal artery or other vessel of a human body allowing the injection an ablative fluid for ablating tissue, and/or nerve fibers in the outer layer or deep to the outer layer of the vessel, or in prostatic tissue. The system may also include a means to limit and/or adjust the depth of penetration of the ablative fluid into and beyond the tissue of the vessel wall. The catheter may also include structures which provide radial and/or lateral support to the guide tubes so that the guide tubes expand uniformly and maintain their position against the interior surface of the vessel wall as the sharpened injection needles are advanced to penetrate into the vessel wall.

Published U.S. Patent Application Document Nos. 2014032042 and 20110022044 (Garabedian) and U.S. Pat. No. 6,064,914 (Trachtenberg) describe a medical assembly and method are provided to effectively treat abnormal tissue, such as a tumor. The target tissue is thermally ablated using a suitable source, such as RF or laser energy. A cooling shield is placed in contact with non-target tissue adjacent the target tissue, and actively cooled to conduct thermal energy away from the non-target tissue. In one method, the cooling shield can be placed between two organs, in which case, one of the two organs can comprise the target tissue, and the other of the two organs can comprise the non-target tissue. In this case, the cooling shield may comprise an actively cooled inflatable balloon, which can be disposed between the two organs when deflated, and then inflated. The inflatable balloon can be actively cooled by pumping a cooling medium through it. In another method, the cooling shield can be embedded within the non-target tissue. In this case, the cooling shield can comprise one or more needles. If a plurality of needles is used, they can be embedded into the non-target tissue in a series, e.g., a rectilinear or curvilinear arrangement. The needle(s) can be actively cooled by pumping a cooling medium through them.

### SUMMARY OF THE INVENTION

A component for use in magnetic resonance image-guided laser ablation has:

a) a one-piece cannula having at least one laser-transmitting fiber fixed thereto;

b) the one-piece cannula comprising a composition having a proximal insertion end and a thermal energy-emitting tip; and

c) Fluid conducting channels fixed to the energy-emitting tip.

The fluid conducting channels have fluid-carrying dimensions sufficient to transport sufficient liquid at 15° C. through the channels to cool both tissue adjacent the channels and the tip during emission from a tissue ablating laser within the thermal energy emitting tip. The composition of the one-piece cannula is at least translucent/transparent to at least 50% of infrared radiation between 900-1200 nm emitted from within the cannula at the thermal energy-emitting tip. The composition of the one-piece cannula should have a melting temperature of at least 150° C. The same fluid channels can be used to improve navigation when using MRI guided interstitial placement of cannula by pumping paramagnetic agents such as dilute gadolinium to vastly enhance the visibility of the cannula, speed placement of the thermal generating tip to the optimal site.

### BRIEF DESCRIPTION OF THE FIGURES

FIG. 1 shows a perspective view of a cannula with fluid conducting channels fixed to its surface.

FIGS. 1A and 1B are images of a Thermal Tool with Cladding Removed To Show Internal Features.

FIGS. 2A and 2B are images of a Thermal Tool.

FIG. 3A is an image of a robotic thermal tool with the casing of robot removed for illustration.

FIG. 3B is an image of a Direct Steering Mechanism of Central Thermal Tool.

FIG. 3C is an oblique view of the direct steering mechanism of the central thermal tool in the unengaged and undeployed state.

FIG. 3D is an image of a deployed thermal tool.

FIG. 4A is an image of a preplanned strategy developed using an iterative computer based treatment planning software program.

FIG. 4B is an image of a preplanned strategy developed from the planned surgical navigation trajectory as calculated at the perineal musculature.

FIG. 5 is an image of a Robot Safety Enhanced Transportable, Fast Robot Alignment And Positioning Stand.

### DETAILED DESCRIPTION OF THE INVENTION

In this enabling disclosure, specific dimensions, values and numbers may be used to assure compliance with all degrees of legal and technical requirements. Thee specifics should not be considered as limiting the generic disclosure of technology and the invention provided herein.

A component is enabled herein for a thermal tool, or tissue ablation or cauterizing components for use in magnetic resonance image-guided laser ablation/cauterization/devascularisation. The component may include. By way of non-limiting examples:

a) a one-piece cannula having at least one laser-transmitting fiber fixed thereto;

b) the one-piece cannula comprising a composition having a proximal insertion end and a thermal energy-emitting tip; and

c) fluid conducting channels fixed to the energy-emitting tip;

d) a one piece cannula with a distal quick connect locking mechanism to lock the cannula securely in place to the distal mechanized insertion plate plus at least two stabilizing plates to guide its entry.

the fluid conducting channels having fluid-carrying dimensions sufficient to transport sufficient liquid at 15° C. through the channels to cool both tissue adjacent the channels and the tip during emission from a laser within the thermal energy emitting tip. The composition of the one-piece cannula may be at least translucent (including transparent) to at least 40%, 50% and more of infrared radiation between 850-1300, 850-1200, 900-1200 and 850-1050 nm emitted from within the cannula at the thermal energy-emitting tip. The component may have the composition of the one-piece cannula provided with a melting temperature of at least 150° C.

The channels of the component may be aligned in a parallel linear array for at least 10% of the length of the cannula at the thermal energy-emitting tip and in mass transfer communication with a source of a paramagnetic contrast agent can be pumped through the channels to enhance the visibility of the channels under MRI guidance. The channels may be aligned in a parallel (even though encircling the diameter of the cannula) linear array for at least 5, 10, 15 or 20% of the length of the cannula at the thermal energy-emitting tip such that a paramagnetic contrast agent such as dilute gadolinium can be pumped through these channels to enhance their visibility under MRI guidance such that placement of the cannula is done with fewer repositioning thereby reducing internal tissue bleeding which interferes with visualization of the cannula and accurate placement of thermal energy tool.

The channels of the component may be aligned in the form of revolutions about the cannula in an approximately parallel linear array for at least 10% (up to 100%) of the length of the cannula at the thermal energy-emitting tip such the outer and inner structure of the cannula do not exceed 48° C. such that tissue immediately adjacent to the cannula is prevented from overheating and charring, thereby absorbing energy at the cannula and preventing optimal thermal energy transmitted the maximal distance through tissue for the longest duration of time. The cannula may have a lumen of sufficient dimension to allow entry and withdrawal of a laser emitting fiber having dimensions of between 0.2μ and 2.0μ and capable of transmitting between 4-40 Watts of laser power through the composition without melting the composition. 20. The laser can be adjustable as well as turned on and off. Laser power may be controlled from minimum power draw and output from nearly zero through and up to all levels of power needed to perform the medical procedures. The composition of the one-piece cannula is at least translucent to at least 40%, 50%, 70% (or more, up to transparency) of infrared radiation between 900-1200 nm emitted from within the cannula at the thermal energy-emitting tip and the composition of the one-piece cannula has a melting temperature of at least 150° C. The component may be, and preferably is supported in a robotic surgical insertion and powering device (an automated guidance and motivation system), wherein the channels are in communication with a fluid pump and a source of liquid at a temperature of no more than 15° C. and the cannula has a lumen of sufficient, and within the cannula is a laser emitting fiber having dimensions of between 0.2μ and 2.0μ and capable of transmitting between 4-40 Watts of laser power through the composition and the laser emitting fiber is in communication with a source of infrared radiation.

The component may be supported in a robotic surgical insertion and powering device that is located initially superior to the patient and composed of a central main channel in which cannula is rigidly attached by a quick threaded connector positioned 10 mm from the distal tip to a counter-threaded channel in the plate. The optical fiber and 2 cooling (in and out fluid pathway) exit directly through the back of the plate to be connected to their respective mates. When so directed a worm gear mechanism is activated which elevates or retracts the plate and forces the cannula, which remains aligned in the central position by means of 3 more proximal centrally channeled plates. The robotic surgical insertion and powering device may be located superior to a patient and comprising a central main channel in which a cannula is positionable through a proximal opening without deviation from a straight path by means of a worm gear drive The component supported in a robotic surgical insertion and powering device may be located superior to the patient and composed of a central main channel in which a cannula, if forced forward, is surrounded by a separate peripheral plate which can independent of the central cannula can force an additional 1-3 prepositioned cannulas forward by an independently controlled but similar motor gear plate mechanism. The location of placement of the additional cannulas is a function of anticipated deflection of the central cannula or need for additional illumination laterally or circumferentially. The cannula may have a lumen containing a fixed laser emitting fiber having dimensions of between 0.2μ and 2.0μ and capable of transmitting between 4-40 Watts of laser power through the composition without melting the composition. The channels may be from 0.3 to 0.8 mm in diameter and may be wound at 8-16 revolutions per cm length of the cannula in a combinations of clockwise and counter clockwise directions yielding (in a two channel parallel array) 16-32 revolutions per cm length of the cannula to aid in visibility of the tip during MRI navigation, and cooling of the fiber during energy emission. The worm gear drive may be remotely activated and powered by an MRI compatible motor located at a base of the robotic device. The component may include at least multiple ablating laser sources available for multiple fiber optic transmitters and each laser may be independently powered to enable a conformal zone of destruction. Please reference copending U.S. patent application Ser. No. 14/324,227, filed 6 Jul. 2014 and Published US Patent Application Document no. 20140135790 (Trachtenberg) which are incorporated herein by reference in their entirety. The component may have multiple ablating laser sources available for multiple fiber optic transmitters and each laser is independently powered to enable a conformal zone of destruction. The component may have a pointed tip to assist in skin piercing during positioning. The component may be secured to an automated target alignment device. The automated target alignment device may be seated on an automated platform that allows the automatic target alignment device 6 degrees of freedom.

The technology further may include at least a thermal medical procedure tool comprising a one piece molded translucent PEEK plastic thermal tool with: a) skin piercing conical (5-20 mm long), such as 10 mm long cannula preferably having a sharpened tip, b) moulded or otherwise manufactured to the appropriate shape (0.3 to 1.0 mm, such as 0.5 mm diam.) fluid carrying channels revolving (encircling, with 6 to 40, preferably 8 to 36 and more preferably between 16 and 24 total windings or revolutions/centimeter), for example at 20 rev/cm (one-half or 10 rev/cm in outer channels connected at distal tip to inner channels at, for example 4-20, or preferably about 10 rev/cm) for 4 cm (2-10 cm) about distal portion of laser fiber until tapered tip to enhance visibility by circulating dilute MRI contrast enhancing agents like Gadolinium during tool navigation period and to cool the edges of the tool during illumination to prevent carbonization of adjacent tissue, absorption of illumination, decreased diameter of illumination, c) fixed central (near infrared, between 850 and 1080 nm wavelength) 980 nm laser fiber (0.4 or 0.6 mm diam.) with 1 cm, 1.5 cm, or 2 cm or no distal diffusor tip d) 1 outflow of fluid channels d2) connector for laser fiber d3) inflow of fluid channels. The Thermal Tool measures, for example, 2.6 mm in O.D. by, for example, 90 mm in length plus an approximately 10 mm conical distal tapered tip

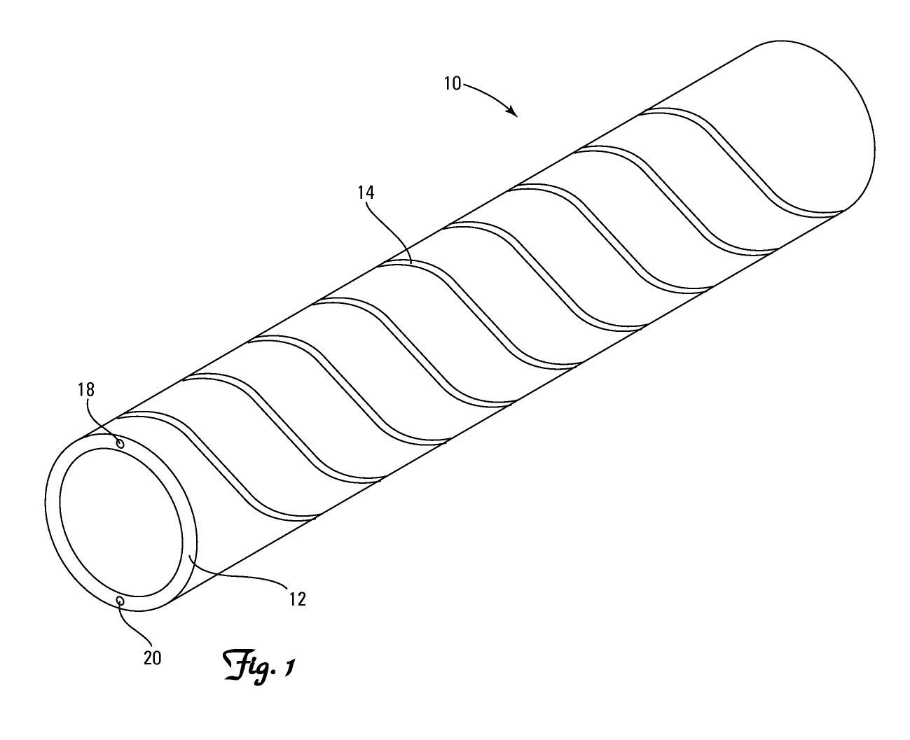

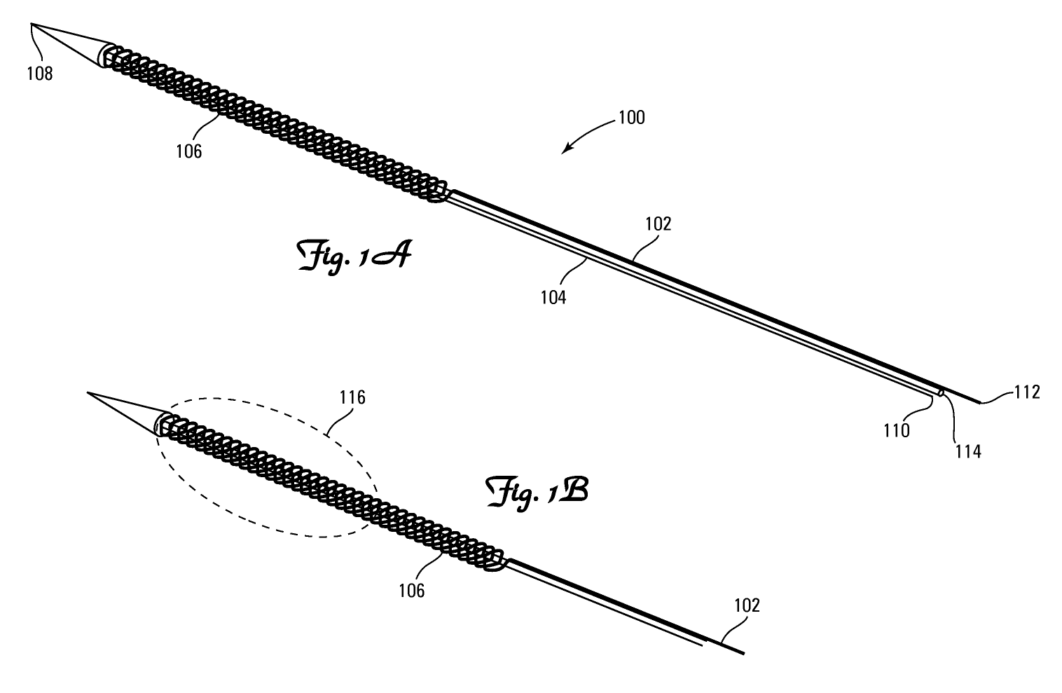

FIG. 1 shows a perspective view of a cannula 10 with fluid conducting channels 14 fixed to its surface. The Channels 14 are accessed by fluid entry opening port 18 and fluid exit port 20 that enters through the end 12 of the cannula 10/

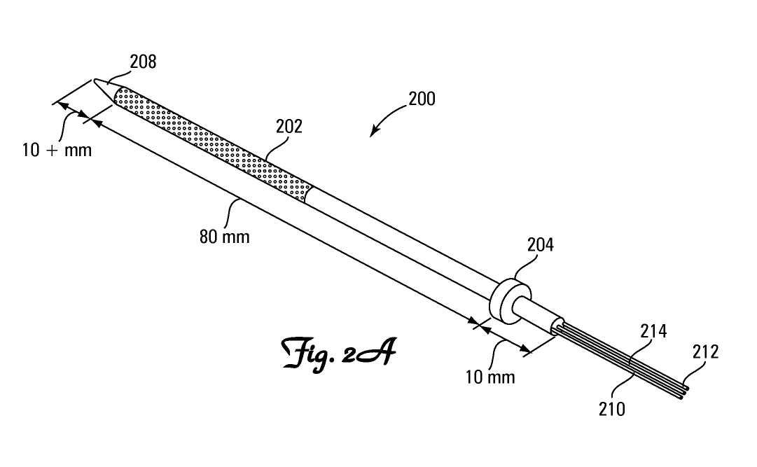

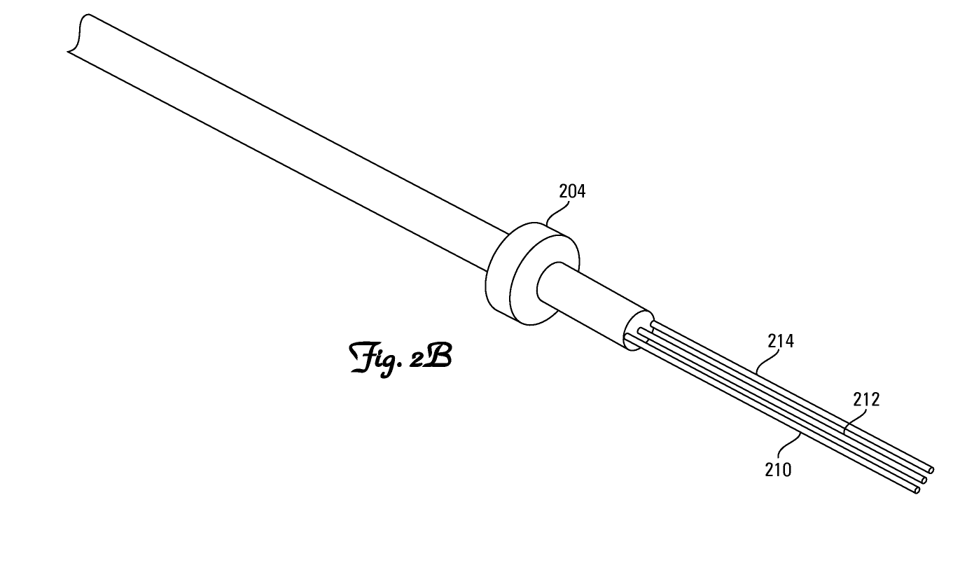

FIGS. 2A and 2B are images of a Thermal Tool with cladding and showing a) quick connector hub lock mechanism to tool pusher plate 10 mm from proximal end of tool and illustrating connecting ports exiting from end of tool to exit base plate and then to through robot casing and then externally connected b) central laser fiber to diode laser source (0-30 W, 940-1064 nm, preferable 980 nm) and source of c) inflow and e) exit of the roller pump induced variable flow and pressure controlled fluids to fill the moulded channels with fluid as in FIG. 1a, designed to enhance MRI visibility during navigation (0.1% Gadolinium solution) of the tool to the target and cooling during illumination of the central laser fiber to prevent overheating, charring, absorption of illumination, and thereby decrease the diameter of potential Illumination and thermal destruction. The thermal tool measures 2.6 mm in O.D. by 90 mm in length plus 10 mm sharpened, conical skin piercing tip. FIG. 2 b Threaded hub 10 mm from proximal end of the thermal tool allows for quick connect and disconnect from the rigid hold of the robot's reverse threaded pusher plate connector hole.

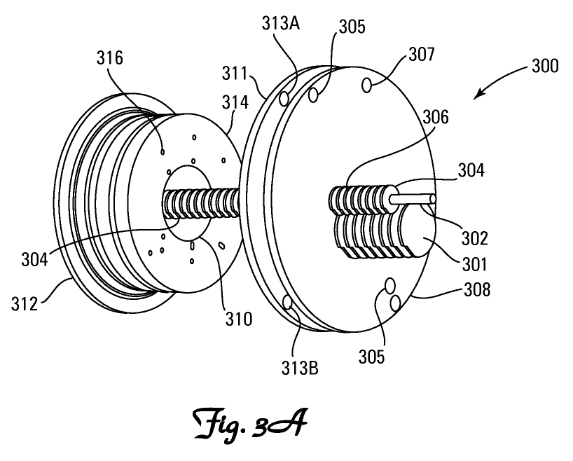

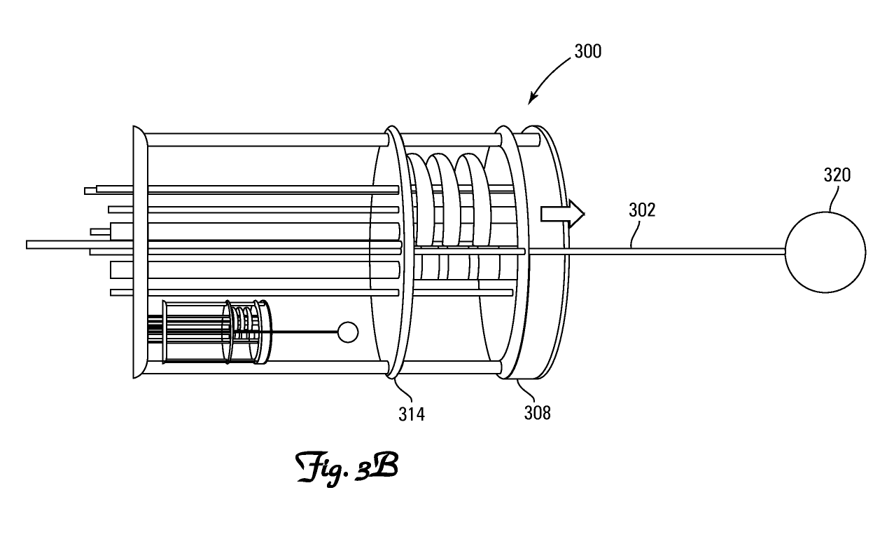

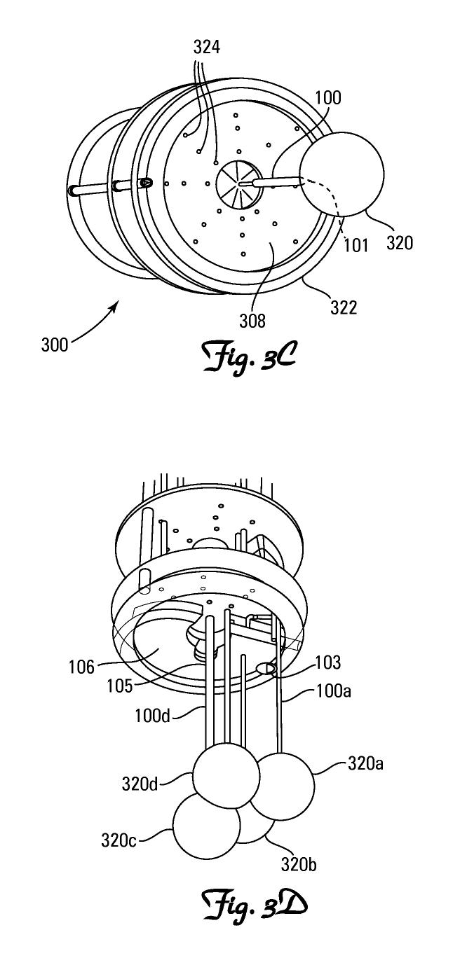

FIG. 3A is an image of a robotic thermal tool with the casing of robot removed for illustration. The Thermal Tool is partially deployed a) locked in Central Position of automated target alignment device (Robot) with b) quick lock hub connector attached to c) central motorized pusher (insertion) plate. The tool is stabilized in its path by d) 3 locator plates. The pusher plate is moved forward by a e) central worm gear propelled by its own 0 Nano-motor (MRI compatible) fixed to the m) base-plate. A similar motor-worm gear apparatus controls the excursion of the peripheral pusher plate and the insertion and withdrawal of the auxiliary peripherally mounted thermal 1-3 tools (not shown) The “robot” provides 6 degrees of freedom to thermal tool excursion with g) pitch (+/−30o), h) yaw (+/−15o), rotation +/−180o (not shown, external to case), j) up-down (+/−2 cm), k) right-left (+/−2 cm) l) forward-backward (80 mm). FIG. 3b, c. The robot also provides up to 10o of direct tool steer ability via face plate direct pressure thruster plate. All processes are treatment planned, computer assisted movements via Nano-motor and are externally monitored, controlled, and every movement is executed only by permission of the surgeon via a “manually pressure activated operator's dead man stick” which “by default” stops all motions or activities if not continuously activated and in known or potentially unsafe conditions the default stop state is automatically engaged, (such as out of boundary excursion of the thermal tool(s)).

FIG. 3B is an image of a Direct Steering Mechanism of Central Thermal Tool.

Face-on view of the undeployed thermal tool with a) pressure plate not engaged. Note as plate is pulled and rotates from its resting unengaged, not obstructing, unengaged position towards opposing slot in undersurface of face plate and into channel in the lateral structural tube of the base and face plate towards its opposing face's edge by an d) automated pulley connected to the sagittal and medial surface of transverse mounted pressure plate which b) rotates in an off central axis direction against the side of the moderately flexible thermal tool which is held rigidly c) the capture and engagement channel of the distal central stabilizing plate which lies immediately below the face plate which is at the perineal skin surface, the force exerted against the thin 2.6 mm thermal tool causes angulation of the tool of up to 10o relative in the transverse plane where the tool exits its 2.7 mm vertical canal in the secondary plate of the steering mechanism 3 mm below the distal pressure plate's capture and engagement channel whose oblique direction and narrowing channel captures the thermal tool in its vertical axis and by lateral pressure exerted obliquely by the obliquely applied pressure in the superior portion of 3 mm space between the resisting inferior plate's vertical channel and the pressure applied by the pressure plates sweeping movement causes an off axis deviation of 5-10o of the thermal tool relative to the vertical axis on exit from transverse face plate of robot. The robot can rotate +/− 180o by external movement of its case (not shown) allowing for potential angulation in any direction. (a) is unengaged pressure plate; b) is rotation point of plate; c) is the obliquely directed thermal tool capture and engagement channel of the distal stabilizing pressure plate; d) is the pressure plate pulley attached to the sagittal surface of the pressure plate and exerts a transverse and oblique pressure on the superior aspect of the 3 mm space which the tool traverses before exiting the robot's face plate and thereby causes an angulation of 5-10o off the initial vertical axis in the direction of the pulley which exits though a canal in the lateral structural tube of the robot and is engaged by a nano motor controlled spooling mechanism located on the base plate and externally controlled.)

FIG. 3C is an oblique view of the direct steering mechanism of the central thermal tool in the unengaged and undeployed state.

Oblique view of description in FIG. 3b, a) is the unengaged pressure plate, b) is the rotation point of pressure plate c) is the capture and engagement channel of the pressure plate d) is the 3 mm space in which the lateral and superior pressure on the thermal tool causes it to angulate off axis 5-10o.

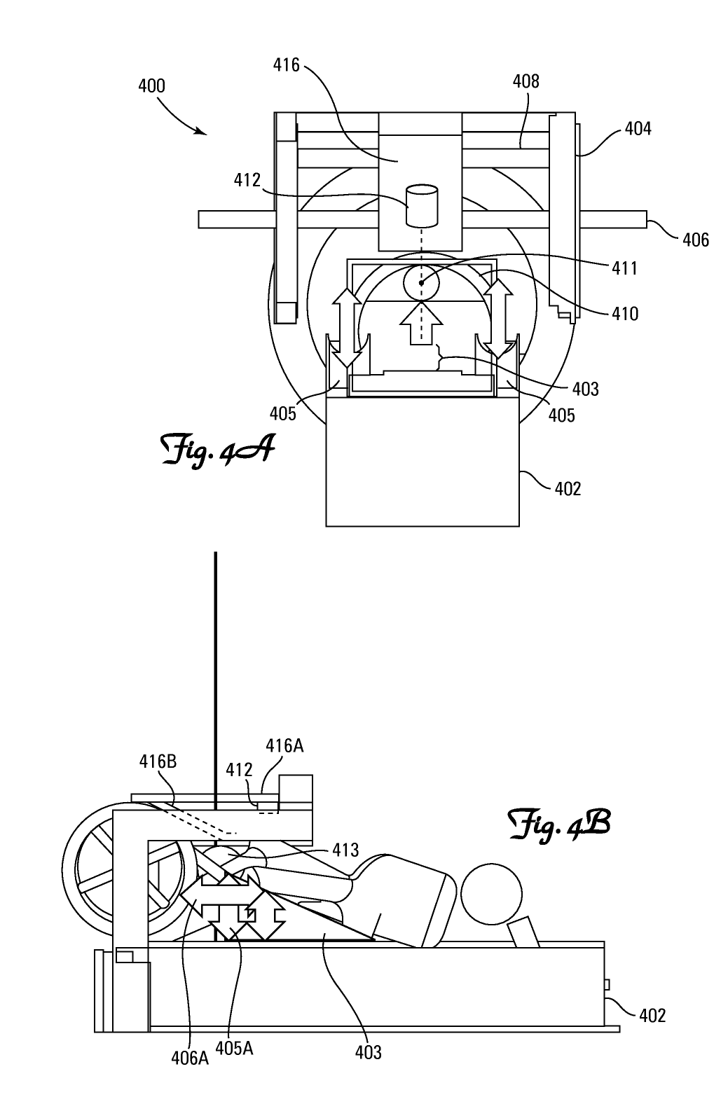

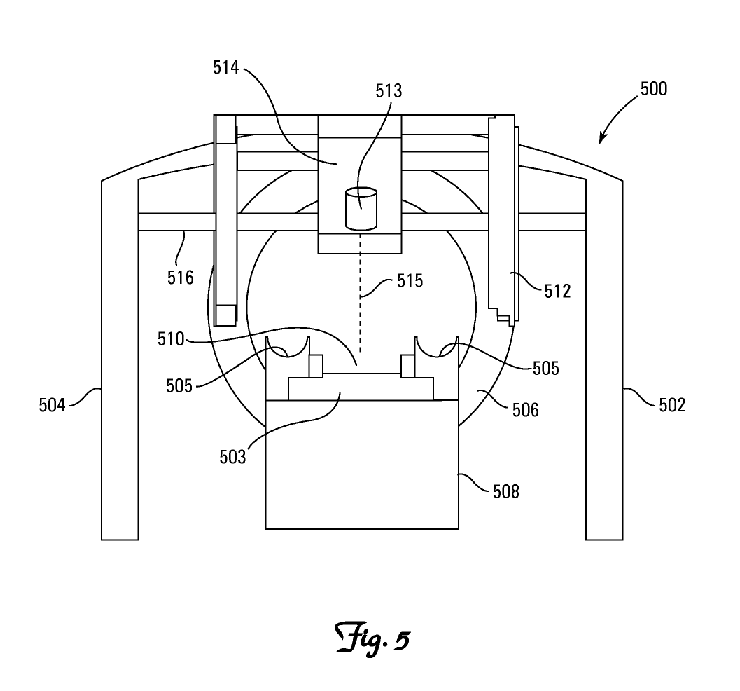

FIG. 4A) shows a visualization of the thermal tool is performed using a devoted custom developed MRI sequence, FSGER (fast spoiled gradient echo recalled) on a 1.5 T GE MRI scanner capable of producing images at real time speed (0.2 seconds per image). The yellow line is the projected trajectory of the thermal tool to the tumor target within the prostate based on the treatment planning software.

FIG. 4B) If the deviation of the thermal tool from the planned surgical navigation trajectory as calculated at the perineal musculature is <10 o for a “near” tumor located at the distal portion of the prostate requiring minimal extension of the thermal tool (<45 mm thermal tool extension e.g., for a distal apical tumor, or distal peripheral zone tumor) or <5 o for a tumor requiring maximal extension of the thermal tool (>70 mm thermal tool extension, e.g. adjacent to the bladder, anterior basal tumor), the thermal tool is retracted to a position just within the body beyond skin surface, and the steering plate is engaged against the thermal tool in the opposing direction of the deviation at its proximal position. The tool is extended with maximal force of the plate against the thermal tool and carefully monitored to see if the thermal tool can reassume the planned navigation path to the optimal site within the tumor target.

FIG. 5. Robot Safety Enhanced Transportable, Fast Robot Alignment And Positioning Stand.

FIG. 5b shows a neutral horizontal position of the robot stand and platform superior to the patient which, prior to use yields enhanced safe condition, triple registration and alignment technology for added initial speed and precision as well as inter room, inter institution portability to lower over-all cost

a1) Neutral position horizontally assuming robot platform; a2) robot inferiorly attached to platform. b) The platform rotates on two independent articulating axes up to 45o to obtain the registration of the robotic device will still away from the MRI and final fiducial (containing wax incorporated Gd in 4 known cube positions of the robot) visibility and registration of robot to the MRI visible pelvic space fiducial markers. A novel use of targeting and intersecting HeNe laser beams from known anatomic positions in the pelvis to meet similar beams of the descending and rotating robot on its platform speeds registration before even the appearance of fiducial markers, further with the alignment of the elevated prostate from its descending position within the human pelvis to match the angle of robotic descent by elevating the pelvis of the patient and contained downward average −15o directed prostate, upwards by means of: a c) variable height high pressure individually inflatable linear aligned triangular shaped sacral pelvic support pillows (thereby flattening the often off the horizontal coronal axis of prostate to rectum and thus decreasing the risk of inadvertent puncture of the rectum especially in large prostates with lateral based tumors which tend to droop down from a flat horizontal coronal prostate rectal axis presenting a possible bulge of the rectum to the approaching sharp tip of the thermal tool. the flattening of the posterior prostate and the elevating of the prostate apex to the angle of descent of the robot thereby facilitating a rapid straight trajectory for the descending robot; the elevation of the prostatic apex is further aided by the elevation of the independently controlled rise and descent of the thigh supports as well as 60° forward backward opposing movement off the vertical of the thigh supports causing up to 10° rotation of the pelvis thereby maximizing the visibility of lateral based tumors and presenting them in the preferred “straight on trajectory” of approaching thermal tool thereby minimizing the need for robotic angulation of the tool and possible tool deflection. This projection of the robot to the perineum gives maximum operator visibility of the perineal robot skin penetration zone plus ease of axis to the robot if there need to be quick changes to the robot's operations or the repositioning or addition of its thermal tools). d1) Thigh supports that can be individually raised to approximately 4 cm of the inner roof of the MRI bore or lowered to be horizontal with table top; the action of the thigh supports works with c) above to elevate pelvis into the very high lithotomy position to increase direct visibility of the perineal robot penetration site d2) the thigh support also articulate forward and backwards independently (over an arc of 60o) and by causing one to move forward while the other backward, the pelvis and prostate containing tumor can be made to rotate on d3) a circular axis at the iso-center of the MRI magnetic force that the sacrum prostate elevator pivots upon thereby rotating lateral and basal based tumors towards the center of the thermal tool's straight field of approach and thereby enhances the visibility of the target tumor by moving adjacent structures out of the trajectory both to improve MRI VISIBILITY and to facilitate the surgical navigation of the thermal tool along the trajectory to the optimal site for the precise and complete destruction of the tumor target. e) Detachable MRI patient table.

A component for use in magnetic resonance image-guided laser ablation has:

a) a one-piece cannula having at least one laser-transmitting fiber fixed thereto;

b) the one-piece cannula comprising a composition having a proximal insertion end and a thermal energy-emitting tip; and

c) fluid conducting channels fixed to the energy-emitting tip.

The fluid conducting channels have fluid-carrying dimensions sufficient to transport sufficient liquid at 15° C. through the channels to cool both tissue adjacent the channels and the tip during emission from a tissue ablating laser within the thermal energy emitting tip. The composition of the one-piece cannula is at least translucent/transparent to at least 50% of infrared radiation between 900-1200 nm emitted from within the cannula at the thermal energy-emitting tip. The composition of the one-piece cannula should have a melting temperature of at least 150° C.

The component may use a composition for the one-piece cannula that is a synthetic material such as a thermoplastic or thermoset resin. The composition my, by way of a non-limiting example, be a polyethylene ketone plastic.

Ketones are a family of high-performance thermoplastic polymers. The polar ketone groups in the polymer backbone of these materials gives rise to a strong attraction between polymer chains, which increases the material's melting point (255° C. for copolymers (Carbon monoxide ethylene), 220° C. for terpolymers (Carbon monoxide, ethylene, propylene). Such materials also tend to resist solvents and have good mechanical properties. Unlike many other engineering plastics, aliphatic polyketones are relatively easy to synthesize and can be derived from inexpensive monomers, such as with a palladium (II) catalyst from ethylene and carbon monoxide. A small fraction of the ethylene is generally replaced with propylene to reduce the melting point somewhat. Polyketones are noted for having extremely low defects (double ethylene insertions or double carbonyl insertions, and for having a high degree of transparency to radiation, including infrared radiation between 900 and 1200 nanometers.

The component may have the channels formed (molded, extruded, cast, laminated or the like) into the composition and may have a diameter, by way of non-limiting example, between 0.2 and 2.0 mm. The channels may be aligned in ways to improve the cooling efficiency of liquid flowing through the channels. For example, the channels may be oriented in a helical orientation for at least 10%, 20%, 40%, 50% or more of the length of the cannula, especially as measured from the more distal direction of the cannula. The channels may also be aligned in a parallel linear array for at least 10% of the length of the cannula at the thermal energy-emitting tip. The cannula may have a lumen of sufficient dimension to allow entry and withdrawal of a laser emitting fiber having dimensions of between 0.2μ and 2.0μ and capable of transmitting between 4-40 Watts of laser power through the composition without melting the composition. The composition of the one-piece cannula should be at least translucent to at least 50% of infrared radiation between 900-1200 nm emitted from within the cannula at the thermal energy-emitting tip and the composition of the one-piece cannula has a melting temperature of at least 150° C. The component may be supported in a robotic surgical insertion and powering device, wherein the channels are in communication with a fluid pump and a source of liquid at a temperature of no more than 15° C. and the cannula has a lumen of sufficient, and within the cannula is a laser emitting fiber having dimensions of between 0.2μ and 2.0μ and capable of transmitting between 4-40 Watts of laser power through the composition and the laser emitting fiber is in communication with a source of infrared radiation.

The technology may be further described as a one piece molded cannula for MRI guided focal laser prostate cancer ablation (or any other small volume organ with localized cancer). The cannula may have a pre-embedded, central fixed location 600-1000 micron laser fiber capable of transmitting 5 to 30 W of laser derived power (the actual size of the fiber can be variable) surrounded at its distal thermal energy emitting tip by fluid channels molded or fused or laminated directly into or onto the translucent polyethylene ketone plastic (a high temperature, biocompatible, resistant material). These channels may be from 0.2 to 1.0 mm, preferably 0.4 to 0.6 mm, or about 0.5 mm in diameter. These channels may be formed with a configuration and shape wherein the channels revolve at 10 revolutions per cm in a clockwise and/or counter clockwise direction yielding 20 revolutions per cm (surrounding the light emitting laser fiber and cooling the fiber's surrounding potentially thermally elevated interstitial cannula and immediately adjacent tissue, e.g., FIG. 1) and meeting at the distal tip off the cannula's linear portion and exit in a straight line proximal to the light emitting zone. This leaves the cannula at its proximal tip, where it may be connected via Luer lock type connectors to long flexible tubing which ultimately forms a circuit meeting in the MRI control room. There a standard roller pump forces various fluids through the tubing at a controlled speed. The propulsion of fluids through these molded channels in the interstitial cannula may serve three distinct purposes.

1) These channels enhance the MRI visibility of the cannula carrying a laser fiber along its planned interstitial prostate (or other tissue) navigation course to the tumor target during focal prostate laser thermal ablation. Although not being limited to the description of technology, the increased visibility is derived by forcing fluid by means of a roller pressure pump located in the MRI control room connected via long tubing which themselves connect to the entry and exit ports of the channels of the cannula. The flexible tubing connects with Luer lock like quick connectors, to the extensions of the channels and then through a wave guide (hole) into the control room where the variable speed pump is located. The fluid may comprise dilute gadolinium saline solution or similar MRI contrast or paramagnetic agent which is forced through the preformed molded channels which form a closed circuit in the cannula. The MRI scanner effect on the gadolinium solution or similar MRI contrast agent solution causes a marked increase of the gadolinium saline solution visibility within the channels of the cannula and hence the cannula and its contents as well. This effect is what allows MRI to image tissue, it causes a shortening the T1 relaxation time of protons located nearby in the solution. T1 shortens with an increase in rate of stimulated emission from high energy states (spin anti-aligned with the main field) to low energy states (spin aligned). Thermal vibration of the strongly magnetic metal ions in the contrast agent creates oscillating electromagnetic fields at frequencies corresponding to the energy difference between the spin states (via E=hν), resulting in the requisite stimulation and enhanced image acquisition. Enhanced visibility of the cannula, decreases the uncertainty of where the often difficult to see laser cannula actually lies on its path to an also increasingly difficult tumor target. Both cannula and target clear visibility are essential for precise, tumor target destruction limited to only the tumor while preserving from thermal damage adjacent functional tissue to limit side effects. These quality of life diminishing side effects becomes increasingly more common with each insertion or repositioning of the cannula, potential for bleeding, and decreased ability to acquire MRI signal of both the cannula and target. Both effects decrease the likelihood of a successful compete destruction of the tumor. It also dramatically can increase the time to complete the procedure, if possible.

2) Once at the tumor target site, these fluid channels are used to enhance visibility are purged from the tubing and channels and replaced with room temperature saline solution. Forcing this fluid through the cannula's channels cools the surface of the cannula and the adjacent prostatic tissue from the intense laser energy emitted and rapid intense thermal conversion to temperatures that can exceed 100° C. This prevents similar rapid near-field tissue heating, leading to carbonization and charring of this tissue and subsequent absorption of the light energy. This would ordinarily markedly and variably decreases the calculated diameter of illumination, and thus interferes with the planned thermal tissue destruction. Cooling of the adjacent tissues prevents this absorption of energy effect and allows the effective use of an iteratively derived irreversible thermal tissue destruction program which is based on the calculated maximal diameter of unimpeded light passing through specific tissue, its conversion to thermal energy, with a calculated volume of homogeneous temperature increase which decreases with the increasing radius of illumination. Thus the volume of irreversibly thermal induced tissue damage can be calculated and from this a treatment plan generated which suggests the optimal site of laser fiber placement and surgical path to this site to destroy the maximal amount of tumor tissue with the least energy and using the fewest laser fiber insertions.

3) Without cooling, variable charring of the adjacent tissue leads to an unpredictable decrease in the diameter of the laser induced thermal tissue destruction. Lack of a cooled laser cannula has even been associated with catastrophic melting and destruction of even the high temperature resistant materials used in the translucent cannula, not only making the therapeutic procedure impossible to complete as planned but often leaving remnants of melted, charred debris of the cannula and fiber behind within the necrotic prostatic tissue to, at a best case scenario, making accurate re-imaging of the tumor site impossible due to many artifacts of debris left in the field or to a worse case where the debris serves as host to recurrent infection of increasingly antibiotic resistant bacteria which colonize the infected prostate and lay hidden within the interstices of the debris/tissue combination. This catastrophic event has also been reported as the destruction of the distal half of the cannula fiber combination within a melted pool of thermoplastic material, burnt fiber remnants all lying within a pool of necrotic prostatic tissue making removal of these large tissue debris pools impossible except by difficult extirpative whole prostate surgery complicated by scarring and possible irreversible damage to the same tissue which the procedure had been designed to preserve or worse the iatrogenic cause of severe complications such s damage to the muscles of either or both urinary or fecal continence along with a possibility of rectal or urethral injury caused by the initial treatment of a focal lower risk or by its necessary but avoidable salvage therapy,

The device increases precision of laser fibre placement, decreases the MRI imaging time and images required, leading to a decreased work flow, leading to a mean MRI suite time of less than 2 hour from a customary time of 5-8 hours. The cost of the process is thus decreased markedly and is therefore commercially feasible. The following factors are believed to contribute significantly to decreased work-flow MRI time.

4) The one piece device with its fixed position laser fibre illuminating a calculable maximal irreversible tissue damage volume through the use of built in cooling channels is designed to speed work low by decreasing the need for the traditional multiple imaging process steps. These are often caused by the cannula being used as a soft invisible shell for a stiff and visible nonferrous metal (for example, titanium or tungsten) long handled obturator that is forced through the tissue. These may be manually forced with intermittent pressure applied unevenly and for different lengths by an increasingly impatient assistant, who must reach uncomfortably into the bore of the magnet to precisely guide the long handled tight fitting obturator within its soft translucent cannula shell along a precise path to an exact target location. Because of the often narrow bore of the MRI device as well as increasing girth of many patients, visibility of and access to the perineal skin entry site of the cannula/obturator combination with a template based “target alignment system” is both cumbersome as well as difficult to precisely accomplish in a requisite and limited valuable MRI time period. A calculated “best path” surgical navigation route to this site is further made difficult because of the unusual position of the prostate with the midpoint of each of the right and left apex usually sloping 10-20° below the horizontal axis of the midpoint of the base of the prostate (which is immediately adjacent to the bladder) and the position of majority of tumors which lie in the “peripheral zone” of the prostate, immediately superior and adjacent to the corrugated rectal wall, separated from the rectum by 2-3 mm of peri-prostatic fat. The rectum itself is thermally protected by a sensitive system of vascular channels that become quickly hyper dynamic and increase their flow rate from 3-10 times normal in the attempt to protect the rectal mucosa from impending thermal damage. This marked increased circulation in response to rectal warming leads to a heat sink effect and simultaneous but paradoxical cooling of the prostate tissue most adjacent to the rectum, which is the location of the above mentioned majority of prostate cancers, the peripheral zone tumors. The attempted thermal destruction of the common peripheral zone cancer by minimally distant (1-2 cm from the target) non-invasive, or minimally invasive thermal but lacking mm precision in maintaining high voxel temperatures, such as a transurethral rotating sweeping HIFU (High Intensity Focused Ultrasound) delivered with a sweeping thermal widening 2 dimensional wave through the prostate elicited by usually <10 HIFU transducers is destined to elicit the exact mechanism which it seeks to abolish, viz, the homeostatic protective mechanism of the rectum whose hyper circulation to protect itself is actually the same mechanism which protects those biologically active prostate cancers just below the prostatic capsule, expanding to the extra-prostatic zones causing local progression and perhaps later metastatic spread. The same phenomenon is seen in trans-rectal HIFU devices both those with minimal numbers of transducers which need to be offset from their second US focal point to achieve accurate and maximum target zone heating. Because of the worry of rectal damage caused by optical mismatching of the transmission site caused by any creasing of the rectal mucosa or site of energy transmission into the prostate, a cooled balloon is inflated into the rectum overlying the multi-transducer arrays. This further complicates and dooms to failure even the most carefully and complex of engineered systems with up to 1000 transduces attempting to link with computer precision the small voxels of HIFU generated thermal areas of damage at the recto prostatic junction. The cooling balloon centrally distorts the prostate upwards while allowing a drooping of the lateral areas of prostate tissue over the edges of the balloon. This makes the transfer of a true target using present day systems that do not display a real time image which is impossible to focally locate and thermally destroy the most common sites of tumor because of the natural hyper circulatory vascular cooling by the rectum to protect itself. In so doing, there is cooling of adjacent structures, which in the case of thermal destruction of peripheral zone prostate, protects the most dangerous type of cancer, those immediately adjacent to the capsule, and about to enter a marked dangerous EPE phase by natural and engineered systems enhance cooling of the rectum and inadvertently the prostate thus protecting the actual site of intended destruction.

Furthermore, the usual system of fixed body registered, fused, pre-treatment diagnostic mp MRI images of prostate and transfer of the diagnostic “real” tumor target site to a virtual tumor target is often distorted significantly by the use of an MRI endo-rectal receiver balloon causing upward displacement of central based tumors and an inferior and lateral displacement of lateral based peripheral tumors rendering 2 stage fusion methods relying on a precise localization of tumor. This is likely to fail unless very large portions of the prostate are destroyed in the belief that somewhere within this mass area (half or minimum a quarter of the prostate lies the true tumor target, leading progressively to the increase in side effects as true focal therapy is abandoned in favor of mass destruction of the prostate. Even as one approaches whole prostate in situ methods of cancer ablation the presence of residual cancer because of non-homogeneous, non-confluent, non-conformal, or non-confirmed destruction of these tumors often leads to rapid clinical and histologic recurrence and worse than primary whole prostate salvage therapy outcomes in both oncologic and HRQoL terms.

The usual trans-perineal MRI image guided biopsy of the prostate or Trans rectal routes described above when converted into focal thermal or radiation means of focal cancer destruction are faced with the basic anatomic difficulties inherent in the location of the prostate sloping from bladder downward about −10 to 15 degrees from the horizontal to perpendicular within the bony pelvis. It contains and is surrounded by structures that if significantly damaged will lead to lifelong important side effects such as impotence, incontinence, and bowel injury. The device described is devised uniquely to balance quality of life as well as oncologic efficacy.

It uses the above described one piece cooled laser fibre that can be used in a fully automated system that recognises the anatomic difficulties in localization, navigation to the tumor target site, precision of thermal destruction of the real time visualized important caner by targeting only the MRI visible “Index Lesion” felt to be the site of increased density of aggressive cells hence becoming visible with high PIRADS mpMRI tumor scoring system on the basis of volume averaging (with PPV of >85%, and NPV of >90% in the hands of experienced MRI prostate imagers), the largest of the foci of cancer (historically, 4-5 times the volume of other sites, the prime site of local progression (85% historically in large tumors), and as well, a possible single site of metastatic disease. Thus this system is based on MRI image guided localization of the important cancer, knowing full well that prostate cancer is multifocal in origin, and there will be prostate cancer cells of indolent nature tumors left within the organ after outpatient treatment as described. The system is designed to minimize the occurrence of intraoperative surgical or anaesthetic problems, to enhance the precision of localization of the Index Lesion, to minimize the time needed to navigate a single 2 mm enhanced visibility MRI compatible laser fiber carrying thermal destructive tool by routes used to access lateral or bladder based tumors requires an unusual downward manual movement subsequent manual but varying pressure of the assistant to precisely follow the best surgical navigation path computed to get the source of the thermal energy to a precise 3 dimensionally correct site safely, avoiding the many thermally sensitive but critically important structures that lie within or adjacent to the path to the tumor target by intermittently and manually advancing the template/cannula shell awkwardly because of the poor positioning of the target relative to the approaching obturator/cannula along navigation usually by the guidance by intermittently advancing the device and sequentially imaging.

5) Once the cannula is manipulated into the correct location and verified by a single high resolution scan to be in the proper position, the obturator may be removed and replaced by a 980 nm 6600 micron laser fiber that is encased in a cooling sheath. The area is reimaged to ensure that the placement of the fiber has not moved the cannula. If so, the process is restarted by removing the laser fiber, replacing the obturator and repositioning the cannula into the correct position. This process is repeated until the cannula containing the fibre is appropriately placed. The one piece, one push invention does away with these multiple manipulations.

FIGS. 1A and 1B are images of a Thermal Tool with Cladding Removed To Show Internal Features

The exposed tool is shown as a one piece molded translucent PEEK plastic thermal tool with: a) skin piercing conical 10 mm sharpened tip (6-16 mm), b) moulded or extruded (0.5 mm diameter within the range of 0.3 to 0.8) fluid carrying channels revolving (these are fixed in a wound state, not kinetically rotating) at from about 10 to 40, such as 15-30, preferably about 20 rev/cm (e.g., about 10 rev/cm in outer channels connected at distal tip to inner channels at about 10 rev/cm) for about 4 cm about a distal portion of laser fiber about the tapered tip to enhance visibility by circulating (for example, dilute) MRI contrast enhancing agents like Gadolinium during tool navigation period and to cool the edges of the tool during illumination to prevent carbonization of adjacent tissue, absorption of illumination, decreased diameter of illumination, c) fixed central 980 nm laser fiber (0.4 or 0.6 mm diameter) with about 0.8 cm, 1 cm, 1.5 cm, or about 2 cm or no distal diffusor tip d) 1 outflow of fluid channels d2) connector for laser fiber d3) inflow of fluid channels. The Thermal Tool measures about 2 to 3 mm (such as about 2.6 mm) in O.D. by 90 mm in length plus the about 10 mm conical distal tapered tip. FIG. 1B shows a thermal tool laser fiber with 2 cm diffuser tip showing approximate volume of illumination at 12 W of 980 nm using 0.6 mm fiber

FIG. 1A is Thermal Tool with Cladding Removed To Show Internal Features

Shown is a one piece molded translucent PEEK plastic thermal tool with: a) skin piercing conical 10 mm sharpened tip, b) moulded (0.5 mm diameter) fluid carrying channels revolving at 20 rev/cm (10 rev/cm in outer channels connected at distal tip to inner channels at 10 rev/cm) for 4 cm about distal portion of laser fiber until tapered tip to enhance visibility by circulating dilute MRI contrast enhancing agents like Gadolinium during tool navigation period and to cool the edges of the tool during illumination to prevent carbonization of adjacent tissue, absorption of illumination, decreased diameter of illumination, c) fixed central 980 nm laser fiber (0.4 or 0.6 mm diam.) with 1 cm, 1.5 cm, or 2 cm or no distal diffusor tip d) 1 outflow of fluid channels d2) connector for laser fiber d3) inflow of fluid channels. The exemplified thermal tool measures 2.6 mm in O.D. by 90 mm in length plus 10 mm conical distal tapered tip

FIG. 2A, 2B show a thermal tool with cladding and showing a) quick connector hub lock mechanism to tool pusher plate about 10 mm from proximal end of tool and illustrating connecting ports exiting from end of tool to exit base plate and then to through robot casing and then externally connected b) central laser fiber to diode laser source (0-30 W, 940-1064 nm, preferable 980 nm) and source of c) inflow and e) exit of the roller pump induced variable flow and pressure controlled fluids to fill the moulded channels with fluid as in FIG. 1a, designed to enhance MRI visibility during navigation (0.1% Gadolinium solution) of the tool to the target and cooling during illumination of the central laser fiber to prevent overheating, charring, absorption of illumination, and thereby decrease the diameter of potential Illumination and thermal destruction. The thermal tool measures 2.6 mm in O.D. by 90 mm in length plus 10 mm sharpened, conical skin piercing tip. FIG. 2 B shows a threaded hub about 10 mm from the proximal end of the thermal tool allows for quick connect and disconnect from the rigid hold of the robot's reverse threaded pusher plate connector hole. In FIG. 2B, threaded hub is 10 mm from proximal end of the thermal tool allows for quick connect and disconnect from the rigid hold of the robot's reverse threaded pusher plate connector hole.

FIG. 3A shows a casing of a robot removed for illustration. The thermal tool is partially deployed a) locked in Central Position of automated target alignment device (Robot) with b) quick lock hub connector attached to c) central motorized pusher (insertion) plate. The tool is stabilized in its path by d) 3 locator plates. The pusher plate is moved forward by a e) central worm gear propelled by its own f) Nano-motor (MRI compatible) fixed to the m) base-plate. A similar motor-worm gear apparatus controls the excursion of the peripheral pusher plate and the insertion and withdrawal of the auxiliary peripherally mounted thermal 1-3 tools (not shown) The “robot” provides 6 degrees of freedom to thermal tool excursion with g) pitch (+/−30o), h) yaw (+/−15o), rotation +/−180o (not shown, external to case), j) up-down (+/−2 cm), k) right-left (+1-2 cm) l) forward-backward (80 mm). FIG. 3b, c. The robot also provides up to 10o of direct tool steer ability via face plate direct pressure thruster plate. All processes are treatment planned, computer assisted movements via Nano-motor and are externally monitored, controlled, and every movement is executed only by permission of the surgeon via a “manually pressure activated operator's dead man stick” which “by default” stops all motions or activities if not continuously activated and in known or potentially unsafe conditions the default stop state is automatically engaged, (such as out of boundary excursion of the thermal tool(s)).

FIG. 3B shows a Direct Steering Mechanism of Central Thermal Tool, showing a face-on view of the undeployed thermal tool with a) pressure plate not engaged. Note as the plate is pulled and rotates from its resting unengaged, not obstructing, unengaged position towards opposing slot in undersurface of face plate and into channel in the lateral structural tube of the base and face plate towards its opposing face's edge by an d) automated pulley connected to the sagittal and medial surface of transverse mounted pressure plate which b) rotates in an off central axis direction against the side of the moderately flexible thermal tool which is held rigidly c) the capture and engagement channel of the distal central stabilizing plate which lies immediately below the face plate which is at the perineal skin surface, the force exerted against the thin 2.6 mm thermal tool causes angulation of the tool of up to 10o relative in the transverse plane where the tool exits its 2.7 mm vertical canal in the secondary plate of the steering mechanism 3 mm below the distal pressure plate's capture and engagement channel whose oblique direction and narrowing channel captures the thermal tool in its vertical axis and by lateral pressure exerted obliquely by the obliquely applied pressure in the superior portion of 3 mm space between the resisting inferior plate's vertical channel and the pressure applied by the pressure plates sweeping movement causes an off axis deviation of 5-10o of the thermal tool relative to the vertical axis on exit from transverse face plate of robot. The robot can rotate +/−180o by external movement of its case (not shown) allowing for potential angulation in any direction. (a) is unengaged pressure plate; b) is rotation point of plate; c) is the obliquely directed thermal tool capture and engagement channel of the distal stabilizing pressure plate; d) is the pressure plate pulley attached to the sagittal surface of the pressure plate and exerts a transverse and oblique pressure on the superior aspect of the 3 mm space which the tool traverses before exiting the robot's face plate and thereby causes an angulation of 5-10o off the initial vertical axis in the direction of the pulley which exits though a canal in the lateral structural tube of the robot and is engaged by a nano motor controlled spooling mechanism located on the base plate and externally controlled.)

FIG. 3C is an Oblique View of The Direct Steering Mechanism Of The Central Thermal Tool In The Unengaged And Undeployed State.

The oblique view of description in FIG. 3B wherein a) is the unengaged pressure plate, b) is the rotation point of pressure plate c) is the capture and engagement channel of the pressure plate d) is the 3 mm space in which the lateral and superior pressure on the thermal tool causes it to angulate off axis 5-10o.

FIG. 3D. Thermal tool deployed, but steering option not engaged (a is unengaged pressure plate). Illumination of the target directly ahead of and just prior to computed and monitored conformal laser thermal energy delivery is achieved by direct adjustment of laser power and speed of withdrawal of thermal tool containing cooled laser fiber contained in thermal tool. a) Multiple auxiliary thermal tools mounted on peripheral pusher (insertion) plate but not deployed. b) Space in which pressure on thermal tool causes off axis angulation

The thermal tool is deployed, but steering option not engaged. There is illumination of the target directly ahead of and just prior to computed and monitored conformal laser thermal energy delivery that is achieved by direct adjustment of laser power and speed of withdrawal of thermal tool containing cooled laser fiber contained in thermal tool. a) Multiple auxiliary thermal tools mounted on peripheral pusher (insertion) plate but not deployed. b) Space in which pressure on thermal tool causes off axis angulation

The thermal tool is deployed, but the steering option is not engaged. Illumination of THE target directly ahead and just prior to computed and monitored conformal laser thermal energy delivery IS achieved by direct adjustment of laser power and speed of withdrawal of laser fiber contained in thermal tool.

Also shown is an illustration of illumination of wide central and lateral based, irregular tumor target. Destruction is by direct deployment of 3 additional “auxiliary” thermal tools (4 total) preplaced on a “treatment planning” probability and inserted by peripheral pusher plate into prostate after central thermal tool illumination seen to be inadequate on initial illumination and monitored MRI thermography for single withdrawal conformal destruction of tumor target. Additional 3 thermal tools preplaced about central thermal tool axis but can be quickly adjusted for lateral very wide, irregular tumors, and directly ahead large irregular tumors e.g. large irregular anterior zone tumors, or to compensate for initial wide, thermal tool deflection, not compensated by steering option, with initial tool left in situ and used as stabilizing mechanism. Computer aided treatment planning software adjusts the power of each laser fiber individually and the speed of the withdrawal of the central and peripheral thermal tools separately to achieve a true actively controlled conformal, homogeneous, confluent delivery of thermal energy (>18,000 J/cc tumor target tissue). By raising the temperature uniformly to irreversible tissue damage levels as monitored in 3 dimensional rotatable colorized temperature maps overlaid onto T2w anatomic pelvic images intersecting the “Regions of Interest”, i.e., the tumor target and adjacent function tissue such as neurovascular bundles which control potency, the distal urethral muscular continence zone, urethra, (continence) and the rectum (bowel function) thereby minimizing or eliminating the common but quality of life robbing side effects of conventional prostate cancer whole prostate therapy by completely destroying the tumor target (MRI detectable, “Index Lesions”) while preserving the adjacent functional tissue from thermal damage by the simultaneous monitoring of tissue temperatures within the target zone and preserved zones viewed by real time MRI thermography and actively and adaptively controlling the delivered energy to only the tumor target. Additional automated thermal safety detectors ‘back up” the computer controlled delivery of energy by automatically decreasing or eliminating the laser power (based on the rate of temperature increase as the thermal zone approaches the edge enhanced boundary of the preservation zones, as well as the absolute temperature at predetermined zones outside and within the preserved zone boundary to decrease power by 75% of the specific source of laser energy if the temperature detected within a zone 2 mm outside of the boundary reaches >45° C. and stop the laser powering the specific site of potential thermal damage and increase the flow of cooled fluid through the thermal tool's cooling channels if the temperature reaches >50° C. 2 mm within the boundary to both actively and passively prevent thermal damage to the “to be preserved zones”.

FIG. 4A) all treatments are preplanned using an iterative computer based treatment planning software program. Visualization of the thermal tool is performed using a devoted custom developed MRI sequence, FSGER (fast spoiled gradient echo recalled) on a 1.5 T GE MRI scanner capable of producing images at real time speed (0.2 seconds per image). The yellow line is the projected trajectory of the thermal tool to the tumor target within the prostate based on the treatment planning software.

FIG. 4B) If the deviation of the thermal tool from the planned surgical navigation trajectory as calculated at the perineal musculature is <10o for a “near” tumor located at the distal portion of the prostate requiring minimal extension of the thermal tool (<45 mm thermal tool extension e.g., for a distal apical tumor, or distal peripheral zone tumor) or <5o for a tumor requiring maximal extension of the thermal tool (>70 mm thermal tool extension, e.g. adjacent to the bladder, anterior basal tumor), the thermal tool is retracted to a position just within the body beyond skin surface, and the steering plate is engaged against the thermal tool in the opposing direction of the deviation at its proximal position. The tool is extended with maximal force of the plate against the thermal tool and carefully monitored to see if the thermal tool can reassume the planned navigation path to the optimal site within the tumor target.

If steering of the tool allows it to resume and maintain its planned navigation path as visible by real time 3 dimensional monitoring of its true trajectory, the tool is fully deployed to the target. However, if images obtained at a position just distal to the prostate capsule, show continuing deviation of the thermal tool beyond 2 mm of its proposed target, in spite of maximum pressure being exerted from the steering plate onto the tool. The tool is then fully deployed, in its deviated state but is used as a stabilizing mechanism, and not illuminated. The robot is prepared for immediate insertion of preplanned and placed peripheral plate thermal tools or by quick adjustment of their position based on the measured deviation of the central tool. The peripheral plate tools are deployed to follow a path calculated to compensate for the initial tool's deviation. FIG. 4c shows the maximum calculated angulation of the central thermal tool steering mechanism which against no resistance in air is 15o; with the homogeneous but firm resistance caused by dental gel used in phantom pelvic models, the maximum deviation achievable is 10o The calculated tissue resistance in the multifocal calcified human prostate which is after multiple multi-core biopsy causing haemorrhages, infection, and chronic inflammation is variable and much greater and may limit steering to only 2o. Work Flow is maximized as are the expected results by not repeatedly repositioning the thermal tools, which obscure the MRI visibility of the target as well as the thermal tool (however enhanced they may be) because repositioning of devices each causes more bleeding and makes the final positioning of the tool a matter of great conjecture, rather than certainty. Thus time, frustration, and ultimately causing a decrease in the effectiveness of the MRI thermography which we is the essential factor separating this method from all others viz, the ability to monitor in real time and adaptively, homogeneously, and conformally control the ongoing cumulative effect of the delivery of destructive energy to an MRI defined although putative prostate cancer signature of the most likely to progress to local extension from the prostate and possibly with time and biologic changes to cells capable of metastatic journey from the prostate itself and life and uncontrolled highly symptomatic growth in tissue other than the prostate. The mpMRI detectable “Index Lesions” are volume averaged areas containing high concentrations of aberrant cells observed under MRI stimulation with characteristics that are highly suspicious for aggressive prostate cancer cell aggregations that clinically are usually noted as the largest (X 4-5 volume) of the multifocal lesions, the most likely to be the cause of and be immediately adjacent and contiguous to extra-prostatic extension, and through the group work of Liu and Isaacs by copy number analysis the source of metastases as well (although subject to dispute by other investigators) in human prostate cancer. The PIRADS mpMRI system is the best performing localizing system for prostate cancer in the hands of the experienced imager to date although its PPV range from 45 to 87% although the system negative PV is usually >85% in the hands of the well experienced prostate MRI imager. These images are seen as highly suspicious sets of different MRI sequences, the most commonly used include ADC (most predictive of cancer showing increased cellular permeability), DWC (seen as areas of restricted diffusion), T2w (anatomic deformities noted as black densities), systematic grading of these tumors by validated weighted scoring systems (PIRADS 1 or PIRADS 2 SCORES 4-5/5), which highly correlate with MRI to Ultra Sound fixed body registered multi-segmented prostate images with biopsies of virtual tumor of the real MRI image fused to 3 dimensional Ultrasound images which are then subject to biopsy or the more infrequent, complex, time consuming, and thus more costly directly acquired real time mpMRI guided biopsies of suspicious tissue, as well as classic experimental histologic based 3 mm thin sliced whole mount sections of radical prostatectomy specimens compared to their pre-treatment and immediate post-operative similarly anatomic positioned mpMRI derived images which demonstrate histological aberrant sites of dense aggregations of aggressive higher grade prostate cells occasionally surrounded by, but poorly seen on mpMRI very aggressive infiltrating cancer cells suggesting that the mpMRI images still require additional imaging aid (e.g. experimental real time simultaneous Na Fl PET to mpMRI fusion scanning) to fully define the total sites of aggressive likely to progress beyond the pelvis to the recognized fatal prostate cancer phenotype.

For the moment, the highly suspicious mpMRI series of images obtained in a high resolution 3T or greater diagnostic scan of the prostate defines both the prostate and the tumor target. At the start of treatment, these prostate images are registered through flexible body, prostate auto-segmentation, with subsequent 3 d fused transfer and registration of the diagnostic mpMRI derived tumor target to the usual 1.5 T or 3.0 T wide bore 80+ cm. surgical Interventional MRI device real time images of the treatment phase prostate. In addition, low resolution near real time derived ADC derived target image are obtained and combined with the Virtual tumor target to obtained a merged “practical target”. Since the virtual target is often subject to loss of true target demonstration due to movement from its original undisturbed position caused by pressure of the intruding thermal tool, haemorrhage, inflammation due to manipulation, or cavitation caused by the intense temperature shifts and boiling of intracellular fluids causing shifting of the target In addition, the frequent low resolution images will demonstrate the actual location of the target but target visibility is increasing obscured by bleeding and inflammation thus obscuring of the real time ADC image of the tumor target. The combination of virtual and real images into a practical target speeds the navigation of the thermal tool to the optimal tumor site of thermal tumor destruction. The successful completion of the process thus requires a work flow process that minimizes repositioning of the thermal tool which by necessity causes bleeding, obscures the acquisition of the low resolution ADC image, forcing the determination of the true tumor location by a series of decreasingly useful and time consuming image acquisition sequences, such that often the frustration of the team at the termination of the navigation of the thermal tool (s) to the optimum site is a “good enough” site and may be several or many mm from the true tumor target site. Further decreased visualization of the target also has negative consequences on achieving the goal of procedure; MRI based real time accurate temperature mapping leading often to variable MRI thermography signal not suitable for the precise temperature mapping of complex volumes of varying thicknesses and containing “black holes” created by instilled small volumes of air through the surface.

FIG. 5 is an image of a Robot Safety Enhanced Transportable, Fast Robot Alignment And Positioning Stand.

The stand operates to maximize “Work Flow” (and allow for commercial use by minimizing the procedure to less than 2 hours), speed, as well as, precise navigation of the thermal tool to the site of optimal thermal destruction of the only the tumor target, safely, all in a comfortable, commercially desirable MRI suite time of <2 hours, we have developed a novel, safety enhanced transportable, MRI-robot alignment, and registration in the patient MRI space, and robot positioning device. The patient is sedated on the MRI gantry of the device in an anesthetic equipped anteroom, and is then transported to the MRI suite where the table joins to the device and attached MRI scanner device (either permanently attached for high volume sites or transportable and used only when required in a variety of locations within and outside of a particular institution, increasing cost effectiveness of use). Unique features include a) superior to the patient location of the robot device, which allows for rapid removal of the robot and evacuation and resuscitation of the patient in a rare emergency medical situation. b) rapid pre alignment and registration of the robot to the patient MRI space images by Visual observation and target alignment of the decent of the robot from its inferior position on a platform descending towards the previously computed optimal perineal skin penetration target via surrogate markers, HeNe laser beams from the fixed positions of the cubical arranged fiducial MRI visible markers within the robot to match known positions within the MRI generated image of the pelvis thus registration of robot to MRI space begins even prior to visibility of the robotics MRI visible fiducial markers. C) The device is designed to minimize the need for angulation and steering capabilities of the robot's thermal tools by actively presenting the major tumor target face directly in front of the advancing thermal tools. This is accomplished by elevating the pelvis with its contained prostate from the usual anatomical position of −15o from the horizontal axis during the prostate's descent from adjacent to bladder in the mid pelvis to the first position of penetration where the apex lies deep in the pelvis, correcting for the irregular corrugated junction of prostate and rectum, and maximize the visibility of the distal laterally based tumors by rotating the pelvis 5 to 10 o even within the narrow confines of a 60 cm bore MRI.

A neutral horizontal position of the robot stand and platform superior to the patient, prior to use yields enhanced safe-condition, triple registration and alignment technology for added initial speed and precision as well as inter-room, inter-institution portability to lower over-all cost. This shows a neutral horizontal position of the robot stand and platform superior to the patient, prior to use. The device supports the a) robot (automated and/or distally controlled moving and powering system on the inferior surface of a b1,2 dual, articulating 45o axes system which allow for rotation of the c) platform which is rigidly connected to its own stable stand superior to the patient which itself is rigidly attached to the MRI device, as well. A) The platform is balanced to assume a horizontal plane above the patient within the bore of the MRI device even with the loss of electrical power by the release of 2 independent emergency release bolts which cause the platform to assume its naturally balanced position above the patient, lower the patient thigh supports, and to detach the patient table from the MRI. These features allow for the rapid egress of the patient from the confines of the MRI bore and resuscitation in the rare but potentially life threatening occurrence of a medical emergency from his usually invisible and barely monitored position within the bore of the MRI device. The superior mounting of the robotic target alignment device maximizes visibility of the perineal skin entry point and access to the robot (if necessary to adjust thermal tool location on the pusher plate). d) Registration of the robot to the MRI space is also speeded by 3 independent processes that commence the registration of the robot into the MRI space outside that space by aligning the fixed mounted visible HeNe laser beams of the robot with similar beams from known MRI visible anatomic structures seen in the MRI image of pelvis while the robot is still far away from the perineum. In addition, the pelvis and contained prostate and its tumor are simultaneously elevated and rotated from the normal −15o descending slope from the upper mid pelvis adjacent to the bladder into the deep pelvis of the apical prostate (making positioning of the thermal tool difficult and dangerous as it takes an awkward and dangerous trajectory first down to enter at the apex and then upward risking puncture of the rectum), Elevation of the entire pelvis by raising the thigh supports upward and simultaneously inflating individual sub pelvic pillows corrects for any horizontal misalignment of the lateral edge of prostate to rectum caused by the not shown pressure of endo-rectal receiver's central deforming balloon. In addition rotation of the pelvis of up to 10o in even a 60 cm bore MRI is possible to improve the desired straight forward approach of the robots thermal tools thereby minimizing deflection of the tool from its planned trajectory to the tumor on increased angulation of the tool.

Triple Site, Tumor—Thermal Tool Positioning and Aligning Mechanism

a1) Neutral position horizontally assuming robot platform; a2) robot inferiorly attached to platform. b) The platform rotates on two independent articulating axes up to 45o to obtain the registration of the robotic device will still away from the MRI and final fiducial (containing wax incorporated Gd in 4 known cube positions of the robot) visibility and registration of robot to the MRI visible pelvic space fiducial markers. A novel use of targeting and intersecting HeNe laser beams from known anatomic positions in the pelvis to meet similar beams of the descending and rotating robot on its platform speeds registration before even the appearance of fiducial markers, further with the alignment of the elevated prostate from its descending position within the human pelvis to match the angle of robotic descent by elevating the pelvis of the patient and contained downward average −15o directed prostate, upwards by means of: a c) variable height high pressure individually inflatable linear aligned triangular shaped sacral pelvic support pillows (thereby flattening the often off the horizontal coronal axis of prostate to rectum and thus decreasing the risk of inadvertent puncture of the rectum especially in large prostates with lateral based tumors which tend to droop down from a flat horizontal coronal prostate rectal axis presenting a possible bulge of the rectum to the approaching sharp tip of the thermal tool. the flattening of the posterior prostate and the elevating of the prostate apex to the angle of descent of the robot thereby facilitating a rapid straight trajectory for the descending robot; the elevation of the prostatic apex is further aided by the elevation of the independently controlled rise and descent of the thigh supports as well as 60o forward backward opposing movement off the vertical of the thigh supports causing up to 10o rotation of the pelvis thereby maximizing the visibility of lateral based tumors and presenting them in the preferred “straight on trajectory” of approaching thermal tool thereby minimizing the need for robotic angulation of the tool and possible tool deflection. This projection of the robot to the perineum gives maximum operator visibility of the perineal robot skin penetration zone plus ease of axis to the robot if there need to be quick changes to the robot's operations or the repositioning or addition of its thermal tools). Thigh supports that can be individually raised to approximately 4 cm of the inner roof of the MRI bore or lowered to be horizontal with table top; the action of the thigh supports works with c) above to elevate pelvis into the very high lithotomy position to increase direct visibility of the perineal robot penetration site d2) the thigh support also articulate forward and backwards independently (over an arc of 60o) and by causing one to move forward while the other backward, the pelvis and prostate containing tumor can be made to rotate on d3) a circular axis at the iso-center of the MRI magnetic force that the sacrum prostate elevator pivots upon thereby rotating lateral and basal based tumors towards the center of the thermal tool's straight field of approach and thereby enhances the visibility of the target tumor by moving adjacent structures out of the trajectory both to improve MRI VISIBILITY and to facilitate the surgical navigation of the thermal tool along the trajectory to the optimal site for the precise and complete destruction of the tumor target. e) Detachable MRI patient table.

Numerous variations may be practiced within the scope of the generic present invention. For example, a robot component may be placed on an inferior aspect of a variable angle ramp which descends to meet the angle of the elevated pelvis and its contained prostate. The pelvis and contained prostate I elevated from the prostate mean −15° descent into a +30° angle from the horizontal by motorized thigh supports that can be raised 15-40 cm (e.g., 25 cm) and one moved independently forward while the other is moved backward causing the pelvis to rotate on a central movable plate allowing 5-10° of in bore lateral movement of the pelvis.

Alignment of the component may be assisted by aided by visually guiding the robot from its superior position via Helium-Neon (or other visible light) beam markers before being registered in magnetic resonance imaging space via gadolinium fiducials.

The technology includes a method including initially moving the patient and his prostate tumor (or other organ) into the trajectory of a straight non-angulated cannula, as much as is possible. This is done instead of having the robotic device angulating only the cannula bearing the laser fiber. This initial movement markedly decreases the likelihood of angular cannula deflection (which is magnified as the cannula proceeds deeper into the prostate, both as a function of a maintained angle and distance between the target and the thermal tip of the cannula. The initial movement to reduce any force that increases the angle because of the heterogeneity and irregular firmness of the tissue (e.g., caused by multiple areas of calcification, as by previous biopsies, hemorrhage, or inflammation). Thus it is a unique observation and worthy of some form of disclosure. In the method an object may be to minimize deflection of the incoming cannula by having the cannula as close as possible to an approximately 90° direction of penetration of the skin. Thus, the elevation and slight rotation of the pelvis to align the tumor to the oncoming cannula is an important addition to metgodology. In spite of this, the present technology has added the design of as many degrees of freedom into movement by the robot control (automated direction and power) of the cannula to allow for precise and straight navigation and placement of the tip of the cannula in 1 or 2 attempts maximum. It is also suggested as part of the present technology to use a series of inflated pelvic or sacrum to apex inflatable cushions to flatten the surface of the prostate such that a cannula penetrating near the rectum might not catch an upward corrugation of the rectum and penetrate the rectum causing a fistula.

The present technology also includes conception and reduction to practice an entire system, including more than the cannula structure and the ancillary insertion components. No one has considered the withdrawal of the cannulas as important. In fact, to get a conformal burn, just as performed by a radiation physicist, the treatment performer would need to fashion a series of radioactive seeds of varying lengths and density to perform brachytherapy of the prostate. Using the present technology in combination with the earlier cited Trachtenberg US Patent Application technologies (reference copending U.S. patent application Ser. No. 14/324,227, filed 6 Jul. 2014 and Published US Patent Application Document no. 20140135790), the same medical treatment effects, and improved effects can be accomplished by preplanning the energy required at each position of the laser fiber(s) on withdrawal by mechanically or electronically altering the rate of withdrawal and the individual power to each laser fiber at each position on withdrawal. This will deliver the precise amount of thermally visible energy to each location as seen on MRI thermography maps done in real time and 3D such that we get a conformal tumor target irreversible tissue damage (i.e., the slower the withdrawal and the greater the power of the laser the greater the energy deposited per unit tissue; conversely for an irregular shaped tumor one might start at quick withdrawal at low power for thin tumor sections and slow the withdrawal down and increase the power such that we get a constant deposition of at least 15-18 k J/cc tumor. This function would be computer controlled. This is quite a significant technical achievement because while standard technology relies on the appealing quality of MRI thermography because of the temperature maps one creates and their conversions into irreversible tissue damage, that process is the least consistent and accurate of all aspects of the procedure. It is therefore proposed that automated computer assisted planned energy deposition by the above part of the invention can improve the performance of this type of medical procedure.

The present thermal robotic tool (the optical fiber/optical element ablation system) can be further enhanced in use by an ancillary system for positioning of the patient within the robotic MRI delivery system described herein. The patient positioning system is particularly useful in adjust the angle of the pelvis of the patient by one or more MRI compatible positioning elements located below the body of the patient. Adjustable, bendable, inflatable, torsionable pads or pillows are provided in strategic positions to enable one or both sides of the patient to be independently and controllably adjusted to lift or rotate a side or center of a patient to better position the prostate towards the thermal ablation tool system. As movement of the patient can make the delivery of the thermal tool occur across a shorter path and at a more appropriate angle, especially for the contact angle of the pointed tip against the skin and the proximity of the sharpened tip to the prostrate, both accuracy and speed of the medical task can be improved, as well as adding some level of comfort to the patient. All identical numbers in different figures represent the same elements.

In the associated figures, the MRI cylinder, Foley catheter, endo-rectal and overlying pelvis 8-channel cardiac coil are not shown, The robot platform rotates rapidly above the patient to the roof of the conventional MRI bore of a magnetic resonance imaging system (FIG. 4A, ablation radiation providing element 416; FIG. 5, radiation providing element 514), the table pillows are deflated (FIG. 4A, patient support element 403; FIG. 4B, support including thigh supports 405 and the distance 403 the patient is elevated by the support element; FIG. 5, relatively flat patient support element 503), and the thigh stirrups (FIG. 4A, thigh support elements 405; FIG. 5, thigh support Element 505), are lowered to table height along arrow indicated direction to allow rapid patient egress and resuscitation in a medical emergency.

The normal posterior prostate plane is corrected within the MRI bore to lie in a true mid horizontal axis by multiple individually inflatable, parallel sagittal attached sub-sacral-pelvic pillows (which may provide at least patient yaw-rotation correction and prostate apex elevation of the pelvis by inflation of at least one sub-pillows support (FIG. 5, patient support element 510) and elevation of thigh supports (FIG. 4A, element 405), to an approximate or exact angle of the oncoming, and descending robot platform (FIG. 4A, guide plate element 416; FIG. 4B, guide plate element 416; FIG. 3A, element 311), and low profile robot face (FIG. 3C, element 322; FIG. 4A, guide plate element 412; FIG. 4B, guide plate element 412). The pelvis can be rotated in a transverse axis by applying contra-lateral force to each of the thigh supports (FIG. 4A, thigh support element 405; FIG. 4B, element 406), while the centre of the prostate remains on a swiveling base (FIG. 4A, element 411; FIG. 4B showing directions of movement 403, 405 and 406; FIG. 5, patient support element 510). In this way, posterior lateral tumors which would normally, because of the long distance from and necessary extreme angle of skin puncture required for navigation of the thermal module to the lateral based tumor site, deflection of module off the proposed navigation path is very common and often forces multiple reinsertion attempts. The ability to rotate the pelvis and thereby the tumor even a modest +/−5 degrees because of narrowness of the MRI bore may move the tumor away from obscuring normal or calcified tissue thereby, improving its visibility, bringing its largest dimension into view, and of most importance decreases the extreme angulation of the thermal module at the skin surface puncture site to achieve an on interstitial surgical path to the tumor site and thereby decreases the likelihood of deflection off its optimal path to land in the best site for total tumor tissue destruction, with no damage to adjacent functional tissue, with the least number of laser passes, and with the most efficient thermal energy expended, and finally in the briefest time.

Thus, the initial mechanical movements of the system are to align the robot bearing its centrally located but un-deployed thermal probe (FIG. 3A, element 311) which lies on the inferior aspect of the downward sloping variable angled robot platform (FIG. 5, elements 508 510; FIG. 4A, robotic platform 402; FIG. 4B, robotic platform 402). Since the robot's structure cannot be registered until it is recognized within the bore of the magnet only through its 3 MRI visible fiducial alignment markers (at known sites at corners of an alignment cube within the structure of the robot), the fiducial substance is composed of concentrated gadolinium and oil contained in wells drilled into the structure of the robot. Unique to this device is the ability to begin the alignment process at the very beginning of movement of the robot while it is exterior to the bore of the magnet. This is initiated by elevating and rotating the patient's pelvis and contained tumor (as described above) and visually aligning the now upward pointing prostatic apex and its MRI visible and inked target, computer derived optimal perineal skin puncture site, to the downward sloping robot platform guided by the visible illumination of a low power HeNe marker beam laser concentric to the central thermal probe laser (FIG. 4A, patient target element 411, FIG. 4B, the optical ablation elements shown as a dark line element passing through guideplate 416A) projected towards the perineum skin puncture site. By simultaneous movement (elevation and rotation) of the patient lying within the MRI bore and correcting the angular and lateral decent of the robot landing site by observing the location of the marker beam, the alignment process can begin far ahead of the entry of the robot into the bore of the MRI device and hastens and optimizes this initial registration process. By maximizing the landing of the robot face in a minimally angulated position to the perineal skin the process increases the precision of the module staying on its planned path to the tumor site and decreases the likelihood of deflection.

The posterior prostate is horizontally aligned on its rectal surface in a mid-transverse plane and the prostate apex is elevated by the described inflatable patient table pillow (which may be parallel or patterned alignments of individually inflatable chambers or tubes) and vertically movable thigh supports to raise the entire pelvis (into the high lithotomy position, from approximately −15 degrees to the horizontal to +30 to 45 degrees to the horizontal) and thus to align the prostate apex with the descending robot mounted on a telescoping platform which when it arrives at its final position is fixed in place by pneumatically activated lateral rods. The superior placement of the robot to the patient leaves an excellent view of perineum in case of necessary urgent adjustments of the robot.

The following is a description of an alignment of a tumor relative to the robot thermal tool.

1. Patients thigh supports (T) rise in the vertical (high lithotomy) direction raising the entire pelvis upward such that apex of prostate is about +30o from the horizontal plane (usually lies −15o from horizontal); 2) Thigh supports also move forward and backward. The patient's sacrum lies on a rotating platform that can rotate the pelvis +/−5o thereby bringing the posterior and lateral based tumors into a more perpendicular Axis from skin puncture site and thereby minimizing deflection of thermal tool by angulation at skin surface. The pelvic platform is connected to thigh supports. To rotate the platform, one thigh support moves forward and other thigh support moves backward causing the pelvic platform to rotate about its pivot point. See FIGS. 4B and 403, 405 and 406.
Into Prostate Apex And Then On To Tumor. Canulla, Insertion Should Be Perpendicular To Skin Cannula To Minimize Deflection Of Robot Thermal Tool (B=Bladder, P=Prostate, A=Prostate Apex)

Normal Position Of Prostate Apex—15o From Horizontal As It Descends Into Deep Pelvis

Elevating Pelvis And Contained Prostate +30o From Horizontal By Raising Thigh Supports Rotating Pelvis—10o

The thermal module containing robot is directed into the perineal skin (as close to perpendicular to facilitate penetration of the skin and minimize deflection of the module which is magnified by tumors that lie in the most distal portions of the prostate: tumors in the posterior lateral portion of the prostate), through the perineal muscles, then into the Prostate Apex and then on towards the tumor. The thermal module insertion should be nearly perpendicular to the skin at insertion by appropriate adjustment of the pillows by relative inflation, deflation, movement of the thigh supports in the vertical plane, and forwards and backwards to cause modest rotation of the prostate. These maneuvers decrease the need for robot angulation of the thermal probe relative to the skin to (minimize surface deflection of the robot thermal tool (B=Bladder, P=Prostate, A=Prostate Apex) and subsequent lateral deflection) to achieve the preplanned surgical navigation path. The normal Position Of Prostate Apex si about 15o from horizontal as it descends into the deepest portions of the pelvis.

Elevating Pelvis And Contained Prostate is done by about +30o From Horizontal By Raising Thigh Supports.

In a normal coronal view of the prostate, it descends into the pelvis. The Prostate Apex descends about 15o from horizontal as it descends into a deep pelvis position.

In a normal Sagital view of the prostate, it descends into the pelvis, the prostate Apex descends about 15o from horizontal as it descends into a deep pelvis position.

Pneumatic Plunger

The Robot (FIG. 3A) is a low profile cylindrical motorized MRI compatible device consisting of a transparent 2 piece clamshell cover (not shown) enclosing the sterilizible contents. The robot measures 65 mm in diameter and 115 mm in length. It is composed of a baseplate (FIG. 3A, element 308) on which sit 5 remotely activated MRI compatible ‘nano motors” (FIG. 3A, elements 301, 303, 305, 307, 309). Two of the motors (FIG. 3A, elements 303, 305) propel the lateral insertion plate (FIG. 3A, element 308) by engaging 2 lateral worm gear mechanism (FIG. 3A, elements 313A, 313B) which travel through threaded elements of the insertion plate to the face plate in a bearing containing end hub. Rotation of the worm gear causes the threaded insertion plate to move forward or backwards. Similarly, 2 central baseplate motors and their worm gear mechanisms (FIG. 3A, elements 306, 304, 301, 302) mechanisms propel a central insertion plate which is initially in the same plane as the peripheral plate. The central plate has attached to its quick connect central port a rigidly connected single thermal tool whose fluid channels inlet and outlet ports as well as the cladded laser connector exit through the base plate to be connected respectively to inlet and outlet connectors of the fluid pump kept in the control room and the connector to the main thermal laser When activated the central plate motors propel the central plate forward or backward and its attached thermal module a variable distance of 0 to 90 mm. The thermal modules are kept along a strictly horizontal path by being forced through 3 precision drilled guide plates (FIG. 3A, elements 315A, 315B, 315C). Once activated the insertion plate forces the central thermal module out of the robot which is held rigidly in the horizontal axis by its guide plates. The thermal module's sharpened tip (FIG. 3C, element 101) pieces the skin and enters the subcutaneous tissue and perineal musculature along a predetermined navigation path. Its path is followed by real time MRI to ensure it follows the predetermined navigation path. If minor deviation is noted (<5>2 degrees off path at the perineal muscles) for a distal tumor, the thermal module is retracted to just inside the skin puncture and the steering plate engaged (Figure D, element 101). This plate forms the last of the three guide plates. It is semicircular in shape with a widened central aperture and an obliquely oriented notch. It is rigidly connected to the robot face only on one side by a hinge. (FIG. 3D, element 103) and on the other side by a nylon cable (FIG. 3D, element 102) directed across the face plate to enter a channel to the base plate where tension on the cable is controlled by the fifth baseplate motor. When the nylon cable attached to the steering plate is tensioned, the thermal module is engaged by the lateral directed plate and trapped it in its oblique notch (FIG. 3D, element 105) which is designed to apply a contralateral force needed to correct deviation. The central insertion plate is again activated and force correction are applied by the steering plate and again the thermal module is followed by MRI imaging as it penetrates the tissue to see if it stays on its predetermined path. If the thermal module continues to deviate >5 degrees laterally off course, it is presumed not salvageable, and it is then fully extended to the junction of the prostate and bladder to serve as a stabilizing bar. The degree of deviation is calculated and the peripheral plate is loaded with one to 3 individually controllable and angled in such a way as to correct for the deviation of the first probe.

An 80 mm motorized horizontal forward and back movement with 2o active steer-ability of 2 mm one piece molded stiff cannula incorporating skin piercing sharpened distal tip of translucent plastic, 940 nm laser fiber with 2 cm diffusor tip fixed 5 mm proximal to tip, multiple fluid cooling channels, and Luer lock like proximal end for rapid fixation to pusher plate, laser fiber and fluid channel connectors exit directly through the base plate.

The active steering mechanism, with a motor to control yaw may include at least 2 cm. Right/Left movement, at least 2 cm. Up/Down movement, at least 8 cm. forward and backward cannula thrust with 2o steer-ability, 30o Pitch control, 45o Yaw control and 360o rotation by a non-illustrated robot cylindrical housing.

The cannula steering plate on the ablation delivery cannula may have the cannula steering plate used only if necessary to correct minor angular deviation.

A thin, small cable, preferably one compatible with MRI usage, such as polymeric cables, such as nylon cable is attached to steering plate, descending in lateral channel to back plate motor to put pressure on the steering plate to counter deviation of the cannula at face plate.

A polymeric (e.g., polyamide or nylon) cable may be attached to steering plate, descending in lateral channel to back plate motor to put pressure to counter deviation of the cannula at face plate.

What is claimed:

1. A component for use in magnetic resonance image-guided laser ablation comprising:
a) a one-piece cannula having at least one laser-transmitting fiber fixed thereto;
b) the one-piece cannula comprising a composition having a proximal insertion end and a thermal energy-emitting tip; and
c) fluid conducting channels fixed to the energy-emitting tip;
d) a one piece cannula with a distal quick connect locking mechanism to lock the cannula securely in place to the distal mechanized insertion plate plus at least two stabilizing plates to guide its entry; and
e) the fluid conducting channels having fluid-carrying dimensions sufficient to transport sufficient liquid at 15° C. through the channels to cool both tissue adjacent the channels and the tip during emission from a laser within the thermal energy emitting tip.

2. The component of claim 1 wherein the composition of the one-piece cannula is at least translucent to at least 50% of infrared radiation between 900-1200 nm emitted from within the cannula at the thermal energy-emitting tip.

3. The component of claim 1 wherein the composition of the one-piece cannula has a melting temperature of at least 150° C.

4. The component of claim 2 wherein the composition of the one-piece cannula has a melting temperature of at least 150° C.

5. The component of claim 4 wherein the composition of the one-piece cannula comprises a thermoplastic or thermoset resin.

6. The component of claim 5 wherein the composition comprises a polyethylene ketone plastic.

7. The component of claim 1 wherein the channels are formed into the composition and have a diameter between 0.2 and 2.0 mm.

8. The component of claim 1 wherein the channels are aligned in a helical orientation for at least 10% of the length of the cannula.

9. The component of claim 7 wherein the channels are aligned in a helical orientation for at least 10% of the length of the cannula.

10. The component of claim 9 wherein the channels are aligned in a helical orientation for at least 10% of the length of the cannula.

11. The component of claim 10 wherein the channels are aligned in a parallel linear array for at least 10% of the length of the cannula at the thermal energy-emitting tip.

12. The component of claim 10 wherein the channels are aligned in a parallel linear array for at least 10% of the length of the cannula at the thermal energy-emitting tip such that a source of fluid paramagnetic contrast agent is attached to a pump so that the fluid paramagnetic contrast agent is configured to be pumped through the channels to enhance visibility of the cannula during interstitial surgical navigation before use of the thermal energy producing laser.

13. The component of claim 10 wherein the channels are aligned in a parallel linear array for at least 10% of the length of the cannula at the thermal energy-emitting tip and in mass transfer communication with a source of a paramagnetic contrast agent can be pumped through the channels to enhance the visibility of the channels under MRI guidance.

14. The component of claim 10 wherein the channels are aligned in a parallel linear array for at least 10% of the length of the cannula at the thermal energy-emitting tip such that a paramagnetic contrast agent such as dilute gadolinium can be pumped through these channels to enhance their visibility under MRI guidance such that placement of the cannula is done with fewer repositioning thereby reducing internal tissue bleeding which interferes with visualization of the cannula and accurate placement of thermal energy tool.

15. The component of claim 10 wherein the channels are aligned in a parallel linear array for at least 10% of the length of the cannula at the thermal energy-emitting tip such that a paramagnetic contrast agent such as dilute gadolinium can be pumped through these channels to enhance their visibility under MRI guidance such that placement of the cannula is positioned fewer than three time thereby causing less internal tissue bleeding and reducing visual interference of the cannula and subsequent MRI thermography.

16. The component of claim 10 wherein the channels are aligned in a parallel linear array for at least 10% of the length of the cannula at the thermal energy-emitting tip and capable of adequate cooling by means of pumped cooled fluid through the molded channels such the outer and inner structure of the cannula do not exceed 48° C.

17. The component of claim 10 wherein the channels are aligned in a parallel linear array for at least 10% of the length of the cannula at the thermal energy-emitting tip such the outer and inner structure of the cannula do not exceed 48° C. such that fibre carbonization is prevented by not overheating and optimal thermal energy transmitted the maximal distance through tissue for the longest duration of time.

18. The component of claim 10 wherein the channels are aligned in a parallel linear array for at least 10% of the length of the cannula at the thermal energy-emitting tip such the outer and inner structure of the cannula do not exceed 48° C. such that tissue immediately adjacent to the cannula is prevented from overheating and charring, thereby absorbing energy at the cannula and preventing optimal thermal energy transmitted the maximal distance through tissue for the longest duration of time.

19. The component of claim 2 wherein the cannula has a lumen of sufficient dimension to allow entry and withdrawal of a laser emitting fiber having dimensions of between 0.2μ and 2.0μ and capable of transmitting between 4-40 Watts of laser power through the composition without melting the composition.

20. The component of claim 11 wherein the composition of the one-piece cannula is at least translucent to at least 50% of infrared radiation between 900-1200 nm emitted from within the cannula at the thermal energy-emitting tip and the composition of the one-piece cannula has a melting temperature of at least 150° C.

21. The component of claim 1 supported in a robotic surgical insertion and powering device, wherein the channels are in communication with a fluid pump and a source of liquid at a temperature of no more than 15° C. and the cannula has a lumen of sufficient, and within the cannula is a laser emitting fiber having dimensions of between 0.2μ and 2.0μ and capable of transmitting between 4-40 Watts of laser power through the composition and the laser emitting fiber is in communication with a source of infrared radiation.

22. The component of claim 1 supported in a robotic surgical insertion and powering device, wherein the channels are in communication with a fluid pump and a source of liquid at a temperature of no more than 15° C. and the cannula has a lumen of sufficient, and within the cannula is a laser emitting fiber having dimensions of between 0.2μ and 2.0μ and capable of transmitting between 4-40 Watts of laser power through the composition and the laser emitting fiber is in communication with a source of infrared radiation.

23. The component of claim 5 supported in a robotic surgical insertion and powering device, wherein the channels are in communication with a fluid pump and a source of liquid at a temperature of no more than 15° C. and the cannula has a lumen of sufficient, and within the cannula is a laser emitting fiber having dimensions of between 0.2μ and 2.0μ and capable of transmitting between 4-40 Watts of laser power through the composition and the laser emitting fiber is in communication with a source of infrared radiation.

24. The component of claim 13 supported in a robotic surgical insertion and powering device, wherein the channels are in communication with a fluid pump and a source of liquid at a temperature of no more than 15° C. and the cannula has a lumen of sufficient, and within the cannula is a laser emitting fiber having dimensions of between 0.2μ and 2.0μ and capable of transmitting between 4-40 Watts of laser power through the composition and the laser emitting fiber is in communication with a source of infrared radiation.

25. The component of claim 5 supported in a robotic surgical insertion and powering device, wherein the channels are in communication with a fluid pump and a source of liquid at a temperature of no more than 15° C. and the cannula has a lumen of sufficient, and within the cannula is a laser emitting fiber having dimensions of between 0.2μ and 2.0μ and capable of transmitting between 4-40 Watts of laser power through the composition and the laser emitting fiber is in communication with a source of infrared radiation.

26. The component of claim 2 comprising a high temperature resistant, biocompatible, molded material and having multiple channels arranged about its proximal thermal tip.

27. The component of claim 13 supported in a robotic surgical insertion and powering device, that is located initially superior to the patient and composed of a central main channel in which cannula is rigidly attached by a quick threaded connector positioned 10 mm from the distal tip to a counter-threaded channel in the plate. The optical fiber and 2 cooling (in and out fluid pathway) exit directly through the back of the plate to be connected to their respective mates. When so directed a worm gear mechanism is activated which elevates or retracts the plate and forces the cannula, which remains aligned in the central position by means of 3 more proximal centrally channeled plates.

28. The component of claim 27 supported in a robotic surgical insertion and powering device located superior to a patient and comprising a central main channel in which a cannula is positionable through a proximal opening without deviation from a straight path by means of a worm gear drive

29. The component of claim 27 supported in a robotic surgical insertion and powering device, that is located superior to the patient and composed of a central main channel in which a cannula configures so that when forced forward, the cannula is surrounded by a separate peripheral plate which can independent of the central cannula can force an additional 1-3 prepositioned cannulas forward by an independently controlled but similar motor gear plate mechanism.

30. The component of claim 2 wherein the cannula has a lumen containing a fixed laser emitting fiber having dimensions of between 0.2μ and 2.0μ and capable of transmitting between 4-40 Watts of laser power through the composition without melting the composition.

31. The component of claim 26 wherein the channels are from 0.3 to 0.8 mm in diameter and are wound at 8-16 revolutions per cm length of the cannula in a combinations of clockwise and counter clockwise directions yielding 16-32 revolutions per cm length of the cannula to aid in visibility of the tip during MRI navigation, and cooling of the fiber during energy emission.

32. The component of claim 28 wherein the worm gear drive is remotely activated and powered by an MRI compatible motor located at a base of the robotic device.

33. The component of claim 1 wherein multiple ablating laser sources are available for multiple fiber optic transmitters and each laser is independently powered to enable a conformal zone of destruction.

34. The component of claim 31 wherein multiple ablating laser sources are available for multiple fiber optic transmitters and each laser is independently powered to enable a conformal zone of destruction.

35. The component of claim 1 wherein the component has a pointed tip to assist in skin piercing during positioning.

36. The component of claim 1 is secured to an automated target alignment device.

37. The component of claim 36 wherein the automated target alignment device is seated on an automated platform that allows the automatic target alignment device 6 degrees of freedom.
